(19) United States
(12) Patent Application Publication
(10) Pub. No.: US 2008/0033341 A1
(43) Pub. Date: Feb. 7, 2008

(54) Methods and devices for reducing or blocking blood flow to a selected blood vessel or part thereof

(75) Inventor: Ygael Grad, Tel-Aviv (IL)

Correspondence Address: Martin D. Moynihan;PRTSI, Inc., P.O. Box 16446, Arlington, VA, 22215, US

(73) Assignee: Bay Holdings Ltd., Tel-Aviv (IL)

(21) Appl. No.: 11/882,813

(22) Filed: Aug. 6, 2007

Related U.S. Application Data
(60) Provisional application No. 60/835,440, filed on Aug. 4, 2006.

(51) Int. Cl. A61B 18/24; A61M 25/00; A61M 29/00; A61M 29/02

(52) U.S. Cl. 604/20; 604/264; 606/194; 606/200

(57) ABSTRACT

A method of reducing or blocking blood to a selected blood vessel or a selected part of the wall thereof, particularly for treating an aneurysm, an arteriovenous or dural malformation in a blood vessel, or for devascularizing tumors, by deploying in the blood vessel an expandable member having a contracted condition for manipulation within the blood vessel, and expandable to an expanded condition in the blood vessel for reducing or blocking blood flow through the selected part thereof, thereby promoting coagulation of blood therein, and for preventing thrombus material from being swept downstream, and applying a local stimulus to the interior of the malformation effective to initiate or accelerate coagulation of blood therein. In some described embodiments, the expandable member is a permeable mesh-like tube of biocompatible material, and in other described embodiments, the expandable member is an inflatable balloon.

### RELATED APPLICATION

This application claims the benefit of U.S. Provisional Patent Application No. 60/835,440 filed Aug. 4, 2006, the contents of which are incorporated herein by reference.

### FIELD AND BACKGROUND OF THE INVENTION

The present invention relates to methods and devices reducing or blocking blood flow to a selected blood vessel or part thereof. This invention is particularly useful for treating aneurysms or other malformations, such as arteriovenous and dural malformations, in blood vessels, and also for devascularizing tumors.

For a brief review of the background to the present invention, particularly with respect to treatments of aneurysms, reference is made to Watson U.S. Pat. No. 5,053,006, O'Reilly U.S. Pat. No. 4,735,201, and McCrory U.S. Pat. No. 5,951,599, and also to published Patent Applications US2003/0100945A1 and US2005/0010281A1 in which the inventor of the present application is a joint inventor.

The above references illustrate the known technique of creating a platelet rich thrombus to occlude a pathology of a blood vessel by photochemical injury to the endothelium. They also illustrate the well known technique of treating an aneurysm in a blood vessel by deploying in the blood vessel a permeable mesh-like tube of biocompatible material to bring the opposite sides of the tube to straddle the opposite sides of the aneurysm such as to reduce blood flow to the aneurysm, and thereby to promote coagulation of blood within the aneurysm. Since the blood within the aneurysm is not circulating with the main blood flow, areas of stagnation are created, and the blood in the aneurysm will therefore thrombose.

One of the problems involved in this method of treating aneurysms is the need to accelerate coagulation of blood within the aneurysm. Another problem is the danger of migration of embolic agents from the aneurysm back into the blood stream particularly in wide neck aneurysms. A third problem is that thrombosed aneurysms filled with predominantly red blood thrombus tend to revascularize, which allows regrowth and recanalization, and prevents adequate tissue scarring and healing of the aneurysm pouch and the neck.

Similar problems are involved in treating other malformations in a blood vessel, such as arteriovenous malformations, and dural malformations, and for devascularizing blood vessels in tumors.

### OBJECTS AND BRIEF SUMMARY OF THE PRESENT INVENTION

An object of the present invention is to provide a method of reducing or blocking blood flow to a selected blood vessel or to a selected part of a blood vessel wall, which method is particularly useful for treating aneurysms and other malformations, such as arteriovenous or dural malformations, or devascularizing blood vessels feeding tumors, which method has advantages in one or more of the above respects. Another object of the invention is to provide devices in the form of a kit particularly useful in the foregoing method.

According to one aspect of the present invention, there is provided a method of reducing or blocking blood flow to a selected blood vessel, or a selected part of a wall thereof, particularly for treating an aneurysm, an arteriovenous malformation, or a dural malformation, or for devascularizing a blood vessel feeding the tumor, such method comprising: deploying in the selected blood vessel an expandable member having a contracted condition for manipulation within the blood vessel, and expandable to an expanded condition in the blood vessel for reducing or blocking blood flow through the blood vessel or the selected part of the wall thereof, and thereby to promote coagulation of blood within the selected blood vessel or part of the wall thereof; and applying a local stimulus to the interior of the selected blood vessel or part of the wall thereof effective to initiate or accelerate coagulation of blood therein.

In some described embodiments, the expandable member is a permeable mesh-like tube of biocompatible material dimensioned, when expanded, to reduce blood flow to the selected part of the blood vessel in which the blood coagulation is to be promoted. In other described embodiments, the expandable member is an occluding member such as an inflatable balloon effective, when expanded, to block blood flow.

In the described preferred embodiments, the local stimulus is preferably light energy applied to the interior of the selected part of the blood vessel in which blood coagulation is to be promoted, by an optical fiber having a tip deployed therein. In addition, a light-energy absorption agent, or a biochemical thrombosing agent, may also be applied to the interior of the selected part of the blood vessel, including the neck and all layers of the malformation. Thereafter an optical translucent or transparent field is established before the light energy is applied thereto.

It is contemplated, however, that the local stimulus could also be a pharmacological agent applied locally or systemically, a mechanical tool such as a coil/device alone or in conjunction with a polymeric component inserted into the selected part of the blood vessel, to induce thrombosis.

Also, in the described preferred embodiments, the permeable mesh-like tube, when used, is an expandable tube having an initial contracted state for enabling moving the tube to the site of deployment in the blood vessel, and an expanded state for fixing the tube within the blood vessel.

In one described preferred embodiment, the permeable mesh-like tube, while in the contracted state, is moved through the blood vessel to a position wherein its opposite sides straddle the opposite sides of the aneurysm (or other malformation) in which the coagulation of the blood is to be promoted. This side of the permeable mesh-like tube facing the downstream direction is expanded; the optic fiber tip is deployed into the aneurysm (or other malformation); and then light energy is applied to the optical fiber to initiate or accelerate coagulation of blood therein while the permeable mesh-like tube prevents emboli resulting from the coagulation from moving through the blood vessel in the downstream direction.

In a second described preferred embodiment, the permeable mesh-like tube, while in the contracted state, is moved through the blood vessel to a position wherein its opposite sides straddle the opposite sides of the aneurysm (or other malformation); the optic fiber tip is deployed into the aneurysm by moving the optical fiber between the outer surface of the permeable mesh-like tube and the inner surface of the blood vessel; the permeable mesh-like tube is then expanded to fix it within the blood vessel straddling the aneurysm; and light energy is then applied to the optical fiber to cause its tip to initiate or accelerate coagulation of the blood within the aneurysm, while the permeable mesh-like tube prevents emboli resulting from the coagulation from moving via the tube into the blood vessel.

A third embodiment is described, wherein the permeable mesh-like tube, while in the contracted state, is moved through the blood vessel to a position wherein its opposite sides straddle the opposite sides of the aneurysm (or other malformation); the permeable mesh-like tube is expanded to fix it within the blood vessel straddling the aneurysm; the optical fiber tip is then deployed by moving the optical fiber through the interior of the expanded permeable mesh-like tube and passing its tip through the permeable mesh-like tube into the aneurysm.

In a fourth described preferred embodiment, particularly in narrow neck aneurysms, a compliant occlusion balloon, while in the contracted state, is moved through the blood vessel to a position wherein its opposite sides straddle the opposite sides of the aneurysm (or other malformation); the optic fiber tip is deployed into the aneurysm by moving the optical fiber between the outer surface of the balloon and the inner surface of the blood vessel; the balloon is then expanded to fix it within the blood vessel straddling the aneurysm; and light energy is then applied to the optical fiber to cause its tip to initiate or accelerate coagulation of the blood within the aneurysm, while the balloon prevents emboli resulting from the coagulation from moving into the blood vessel.

In a fifth described preferred embodiment, particularly in narrow neck aneurysms, a compliant occlusion balloon, while in the contracted state, is moved through the blood vessel to a position downstream to the aneurysm (or other malformation) and then expanded to fix it within the blood vessel distal to the aneurysm; the optic fiber tip is deployed into the aneurysm; and light energy is then applied to the optical fiber to cause its tip to initiate or accelerate coagulation of the blood within the aneurysm, while the balloon prevents emboli resulting from the coagulation from moving downstream into the distal blood vessels.

According to another aspect of the present invention, there is provided a method of treating an aneurysm, arteriovenous or a dural malformation in a blood vessel, or devascularizing blood vessels feeding a tumor, by deploying in the blood vessel leading to the malformation or the tumor a temporary occlusion balloon of biocompatible material, and inflating the balloon such as to temporarily stop blood flow to the malformation. Via a center tube in the balloon a light-energy absorption agent, or a biochemical thrombosing agent, is applied to the interior of the malformation including all layers of the wall before advancing a fiber optic with a diffusing tip through the center tube into the malformation. Light energy is then applied as local stimulus to the interior of the malformation while saline is flushed in the gap between the fiber optic and the center tube to provide an optically translucent or transparent field and prevent thermal damage to the arterial wall. Slow deflation of the balloon is then commenced such as to initiate or accelerate coagulation of blood now perfusing the malformation.

The invention is particularly useful for the treatment of brain aneurysms, aneurysms of other parts of the body such as abdominal aortic aneurysms and aortic arch aneurysms, or arteriovenous malformation or dural arteriovenous fistulas or to devascularize a tumor. In brain aneurysms, particularly with a wide neck, it combines stent-flow diversion with photo-thrombosis therapy techniques for this purpose by using minimally-invasive trans-catheter therapy. Thus, the permeable mesh-like tube, or stent, may be delivered to the aneurysm site through a puncture in the groin, and the optical fiber may then be advanced through a microcatheter into the aneurysm.

A light energy absorption agent, such as Rose Bengal or Erythrocyn B, may be administrated (IV) systemically to create an environment for platelet thrombus formation. Alternatively, the agent can be administered locally into the aneurysm or the malformation via a microcatheter before inserting the optical fiber. After insertion of the fiber an agent such as saline can be infused in the gap between the microcatheter and the optical fiber to establish a light transmitting field to the wall of the malformation.

A pulse of coherent laser light at the appropriate wavelength (400-600 nm) is then administrated through the optical fiber to create a platelet thrombus in the aneurysm or the arteriovenous or the dural malformation. The benefit of a platelet thrombus is that it exploits the fact that the patient has heparin “on board” which works on other “parts” of the coagulation cascade.

According to another aspect of the present invention, there is provided a kit for use in reducing or blocking blood flow to a selected blood vessel, or a selected part or a wall thereof, particularly for treating an aneurysm, an arteriovenous malformation, or a dural malformation, or for devascularizing a blood vessel feeding a tumor, the kit comprising: an expandable member having a contracted condition for manipulation within the blood vessel and expandable to an expanded condition within the blood vessel for reducing or blocking blood flow to the selected part of the blood vessel or wall thereof, and thereby to promote coagulation of blood therein; and a local stimulus applicator for applying a local stimulus to the interior of the selected part of the blood vessel, such as to initiate or accelerate coagulation of blood therein.

According to a still further aspect of the present invention, there is provided a microcatheter, particularly useful in such a kit, comprising: an optical fiber having a tip including diffusive surfaces on its lateral sides for emitting light energy laterally around the tip; a catheter tube enclosing the optical fiber for deploying the optical fiber tip into the selected part of the blood vessel in which coagulation is to be initiated or accelerated; and an applicator for delivering to the interior of the selected part of the blood vessel, before the light energy is applied thereto, a light-energy absorption agent via space between the optic fiber and the catheter tube.

Further features and advantages of the invention will be apparent from the description below.

### BRIEF DESCRIPTION OF THE DRAWINGS

The invention is herein described, by way of example only, with the reference to the accompanying drawings, wherein:

FIG. 1 schematically illustrates one method of treating an aneurysm (or other malformation) in accordance with the present invention;

FIG. 2 schematically illustrates a second method of treating an aneurysm in accordance with the present invention;

FIG. 3 schematically illustrates a third method of treating an aneurysm in accordance with the present invention;

FIG. 4 schematically illustrates a forth method of treating an aneurysm in accordance with the present invention;

FIG. 5 schematically illustrates a fifth method of treating an aneurysm in accordance with the present invention;

FIG. 6 schematically illustrates the method of FIG. 2 for treating an aneurysm of a different shape;

FIG. 7 schematically illustrates one manner of applying light energy to the interior of the aneurysm including the neck, and all layers of the aneurysm wall in order to initiate or accelerate coagulation of blood therein; and

FIG. 8 illustrates an optical fiber, including an optical fiber and a light diffusing tip for promoting coagulation of blood within an aneurysm or other part of a blood vessel to coagulate blood therein in accordance with the method illustrated in FIG. 7; and

FIG. 9 schematically illustrates one method of treating an arteriovenous malformation, or a dural malformation, or a blood vessel feeding a tumor in accordance with the present invention;

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

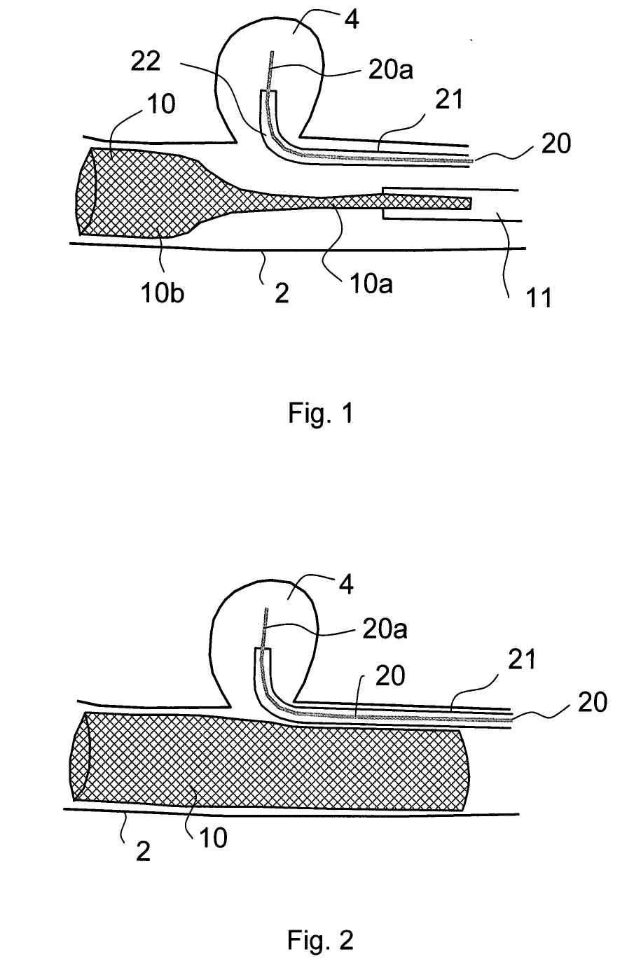

As indicated earlier, the present invention involves a method, and also medical devices, for reducing or blocking blood flow to a selected blood vessel, or a selected part of a wall thereof, particularly for treating an aneurysm malformation, an arteriovenous or a dural malformation, or blood vessel feeding a tumor by using a minimally-invasive procedure to create a platelet rich thrombus in the aneurysm as shown in FIG. 1. The novel method also uses an expandable member to prevent emboli from entering the blood stream. The drawings illustrate several techniques which can be used for implementing the method. While the description below refers to the treatment of an “aneurysm”, it is to be understood that the methods described are applicable to the treatment of other malformations in blood vessels, such as arteriovenous and dural malformations, and also to block blood vessels feeding tumors.

In some described preferred embodiments of the present invention, the expandable member is a permeable mesh-like tube of biocompatible material dimensioned, when expanded, to straddle the selected part of the blood vessel wall such as to reduce blood flow thereto, and also to prevent thrombus from being swept downstream thereof.

In other described embodiments the expandable member is an inflatable balloon effective, when inflated, to block blood flow to the selected blood vessel or selected part of the wall thereof and to prevent thrombus from being swept downstream thereof.

In the latter described embodiments, an arteriovenous malformation, a fistula, or a tumor feeding blood vessel is treated by (1) deploying in the blood vessel the temporary occlusion balloon of biocompatible material such as to temporarily reduce or stop blood flow to the malformation or tumor, and thereby to promote coagulation of blood within the malformation; and (2) applying a local stimulus, preferably light energy, to the interior of the blood vessel feeding the malformation, to initiate or accelerate coagulation of blood therein.

FIG. 1 illustrates an implementation of the method wherein the optical fiber tip is deployed through a microcatheter into an aneurysm after the partial deployment of the permeable mesh-like tube in the blood vessel, whereas FIGS. 2 and 6 illustrate further implementations of the method wherein the microcatheter is deployed into the aneurysm before by full deployment of the permeable mesh-like tube in the blood vessel and the optical fiber tip is then deployed into the aneurysm through the microcatheter.

FIG. 1 schematically shows the blood vessel 2, e.g., an artery in the brain, having developed an aneurysm 4 in a wall thereof. The danger is that the aneurysm will rupture which, if occurring, results in a high rate of death or irreversible brain damage.

The aneurysm is treated by the use of a permeable mesh-like tube 10 having an initial contracted state, as indicated by section 10a, for enabling moving the tube to the site of deployment in the blood vessel, and an expanded state, as indicated by section 10b, for fixing the tube within the blood vessel. Permeable mesh-like tube 10 is made of biocompatible material and is constrained in its initial contracted state by a sheath 11 which, when removed, permits the tube to expand to its expanded state, as well known in the field of stents.

FIG. 1 also illustrates an optical fiber 20 having a tip 20a to be deployed within the aneurysm 4 through a microcatheter 21 for applying light energy to the interior of the aneurysm in order to initiate or accelerate coagulation of blood therein. In FIG. 1, optical fiber 20 is located within microcatheter 21 and is moveable between the outer surface of the mesh-like tube 10, and the inner surface of the blood vessel 2. Alternatively, a pharmacological thrombosing agent can be deployed through microcatheter 21 instead of the optical fiber 20. As will be described more particularly below, optical fiber 20 is spaced from the inner surface of its microcatheter 21 to allow the injection of a light-energy absorption and/or transmission agent into the interior of the aneurysm before using the optical fiber for applying the light energy thereto.

In the described method, the permeable mesh-like tube 10, while in the contracted state as shown by tube section 10a, is moved through the blood vessel to a position wherein its opposite sides straddle the opposite sides of the aneurysm 4. The side of the tube facing the downstream direction, namely tube section 10b in FIG. 1, is first expanded. A light-energy absorption agent is injected via the catheter 21 into the aneurysm. The optical fiber tip 20a is then deployed into the aneurysm. Light energy is then applied to the optical fiber 20 to cause its tip 20a to apply light energy to the interior of the aneurysm while an optically translucent or transparent field is established by infusion of a an optically clear fluid in the gap 22 between the optical fiber 20 and the microcatheter 21. In such a method, the light energy initiates or accelerates coagulation of blood in the aneurysm, while the permeable mesh-like tube 10, particularly its expanded section 10b, prevents emboli resulting from the coagulation from moving through the blood vessel in the downstream direction. Alternatively, a pharmacological thrombosing agent can be deployed through microcatheter 21 instead of the optical fiber 20.

FIG. 2 illustrates a modification in the method, wherein the permeable mesh-like tube 10 is fully expanded before the light-energy absorption agent is injected via the microcatheter 21 into the aneurysm; alternatively, a pharmacological thrombosing agent can be deployed through microcatheter 21 instead of the optical fiber 20, and before the optical fiber is used for applying the light energy to the interior of the aneurysm. The method illustrated in FIG. 2 thus also prevents emboli resulting from the coagulation from moving into the blood stream.

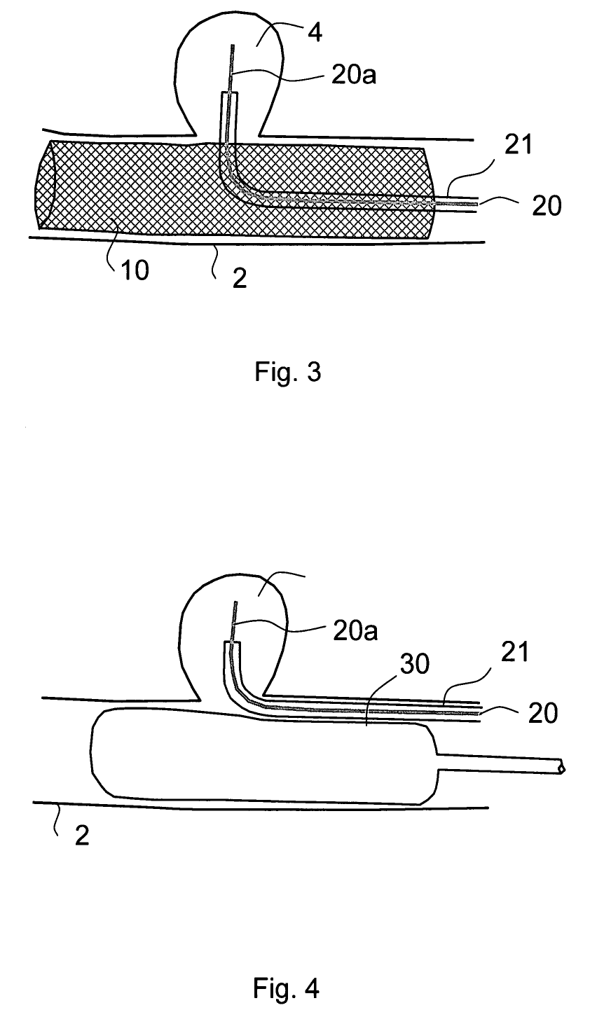

FIG. 3 illustrates a further variation, wherein the permeable mesh-like tube 10 is fully expanded in the blood vessel, straddling the opposite sides of the aneurysm 4, before the optical fiber 20 and its microcatheter 21 are deployed into the aneurysm. In this case, the optical fiber 20 and its microcatheter 21 are moved through the interior of the expanded, permeable mesh-like tube 10, and the tip 20a of the optical fiber 20, is passed through the permeable mesh-like tube into the aneurysm together with the tip of microcatheter 21. Such a variation therefore also initiates or accelerates coagulation of blood in the aneurysm while preventing emboli resulting from such coagulation from moving back into the blood stream. Alternatively, a pharmacological thrombosing agent can be deployed through microcatheter 21 instead of the optical fiber 20.

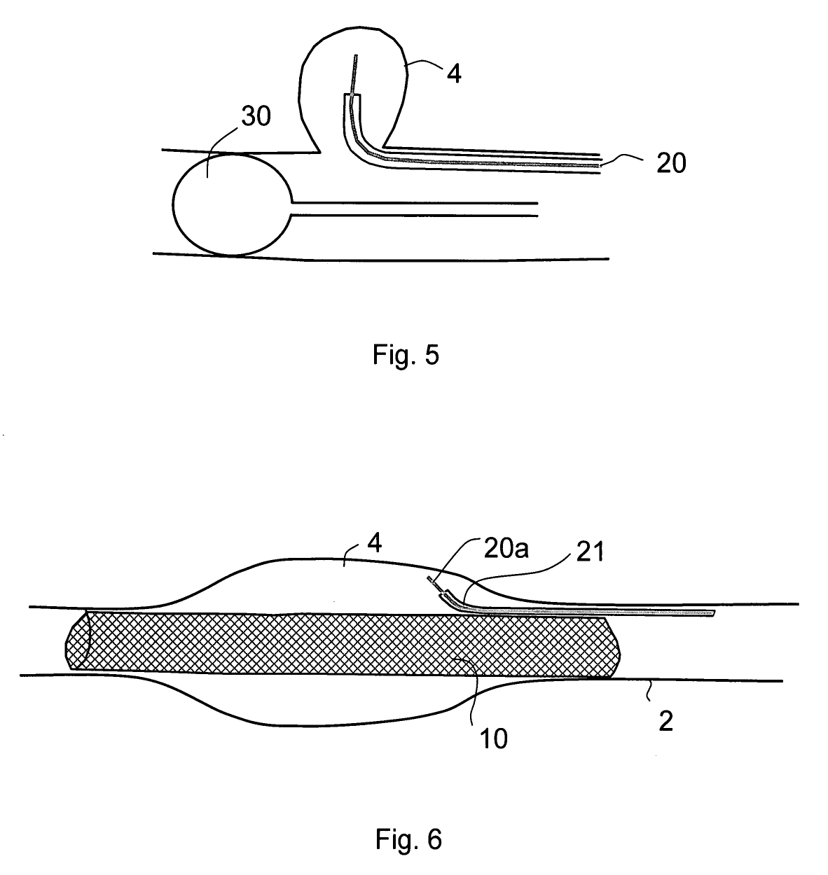

FIGS. 4 and 5 illustrate the novel method implemented by the use of an expandable balloon, rather than an expandable mesh-like tube.

Thus, as shown in FIG. 4, the aneurysm 4 in the blood vessel 2 is treated by deploying a balloon 30 within the blood vessel to straddle the opposite sides of the aneurysm such that, when the balloon is inflated, it occludes or blocks the flow of blood to the aneurysm. It also prevents emboli resulting from the coagulation from moving into the blood stream. In the method illustrated in FIG. 4, the coagulation of the blood within the aneurysm is effected by an optical fiber 20 deployed through the microcatheter 21 in the same manner as described above with respect to FIGS. 1-3.

In FIG. 5, the balloon 30 is deployed immediately downstream of the aneurysm 4 so as to temporarily reduce or stop blood flow to the aneurysm, while a local stimulus in the form of light energy via optical fiber 20, is applied to the interior of the aneurysm to initiate or accelerate coagulation of the blood therein.

In both embodiments illustrated in FIGS. 4 and 5, balloon 30 needs to be expanded only for a very short time until the blood within the aneurysm is sufficiently coagulated, after which time the balloon may be deflated and removed from the blood vessel.

FIG. 6 schematically illustrates a technique similar to that of FIG. 1 or FIG. 2 wherein the aneurysm 4 is of a fusiform shape, so that the permeable mesh-like tube 10 should be of sufficient length to straddle both sides of the aneurysm in the inflated condition of the tube.

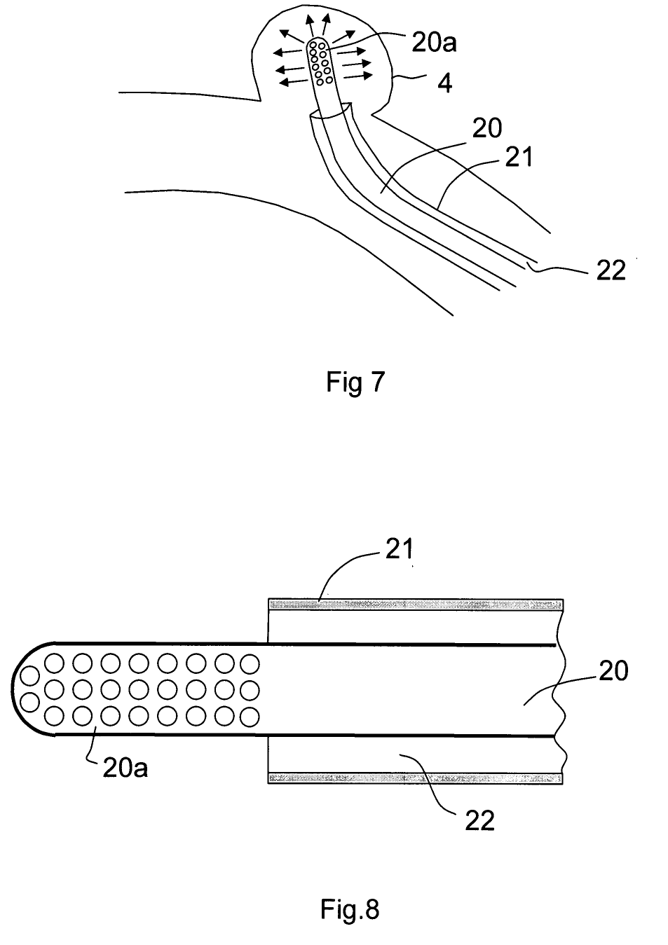

FIGS. 7 and 8 illustrate the distal end of the optical fiber 20, and its microcatheter 21, located within the aneurysm 4. Thus, an annular gap 22 is produced between the optical fiber and the microcatheter for injecting a light-energy absorption agent, or for infusing a fluid to facilitate a translucent or transparent optical field, or for injecting a biochemical thrombosing agent, into the aneurysm before (or during the time) the optical fiber is used for applying light energy (e.g., laser light) to initiate or enhance blood coagulation. It will be appreciated that the construction illustrated in FIGS. 7 and 8 may be used with both the mesh-type expandable member such as shown at 10 in FIGS. 1-3 and 6, or the inflatable-balloon type expandable member as shown in FIGS. 4 and 5.

As shown particularly in FIG. 8, tip 20a of optical fiber 20, includes a convex end cap to semi-spherically disperse light emerging from the distal tip. Tip 20a of the optical fiber further includes light scattering particles, in the form of circular regions 24 which diffuse and distribute the light emitted through the tip and around the lateral sides of the tip. Such a construction produces the light energy to activate the light sensitive dye in the wall, to photochemically damage the endothelium and to initiate or accelerate coagulation of blood therein.

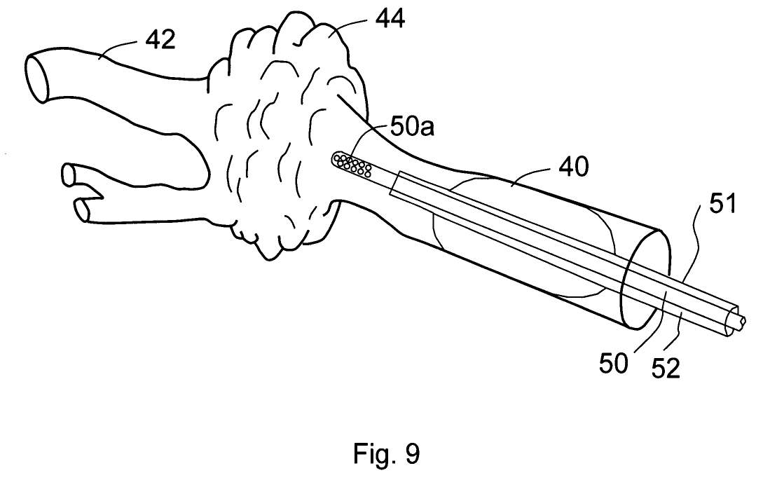

FIG. 9 illustrates the method used for treating an arteriovenous malformation, a dural malformation or a tumor 44, rather an aneurysm. In this application of the invention, the expandable member is preferably a balloon 40 attached to a central passageway microcatheter 51 for receiving an optical fiber 50 having a tip 50a that includes light scattering particles which diffuse and distribute the light emitted through the tip into the malformation or tumor 44. As described above with respect to FIGS. 7 and 8, microcatheter 51 would also include an annular gap 52 between its inner surface and the optical fiber 50 for injecting a light-energy absorption agent, for infusing a fluid to facilitate a translucent or transparent optical field, or for injecting a biochemical thrombosing agent, into the malformation 44 before the optical fiber is used for applying light energy (e.g., laser light) to initiate or accelerate the blood coagulation.

As indicated earlier, preferably a light-energy absorption agent is applied to the interior of the aneurysm sac (or the feeding artery of a malformation, FIG. 9) before the laser energy is applied, so as to make the aneurysm wall and endothelial surface more sensitive to the laser light. This may be done by injecting a light-energy absorption agent via the gap 22 (FIG. 7) between the optical fiber 20 and microcatheter 21. Such a light-energy absorption agent may be, for example, Rose Bengal or Erythrocin B; and the laser light applied may be a pulse of coherent laser light at the wavelength of 400-600 nm. Following are a number of examples of combinations of light-energy absorption agents and laser light wavelengths:

1. Rose Bengal and 562 nm: Peak absorption of light by Rose Bengal is at 562 nm. The laser light is less absorbed by the blood, and therefore it is not necessary to aspire/wash all the blood out of the aneurysm as it can penetrate through.

2. Erythrocyn B+537 nm: Peak absorption of light by Erythrocyn B is at 537 nm. Because of the high absorption of the laser light by the blood, a better washout of the blood from the aneurysm or malformation is necessary for better light penetration through the fluid to the endothelial surface.

The flush of fluid through the gap (FIG. 7) into the aneurysm or the malformation changes the light penetration/absorption coefficient, enabling photochemical damaging of the endothelium, and also absorbs heat energy to prevent thermal damage.

The mechanism of action of the photo thrombosis is believed to be as follows:

a. the dye (Rose Bengal or Erythrocin) is administered into the aneurysm sac or the arteriovenous malformation or the dural malformation;

b. the dye is absorbed by the vascular wall and the endothelial surface

c. a clear optical field is established by infusion of saline in the pathology

d. the dye absorbs the light energy and creates the radical singled oxygen (O₂—released from water containing dye and/or tissue) which is toxic to the endothelial cells;

e. the saline is aspired, and the blood reenters the pathology replacing the saline;

f. the platelets in the entering blood become activated and adhere to the endothelial surface, creating a growing platelet thrombus having a size which depends on the dose of irradiation;

g. the activated platelets that stick to the vessel's wall create a “white thrombus”, which is resistive to anticoagulants such as Heparin usually found in the patient's body during the endovascular procedure.

h. in application to aneurysms, particularly ones with wide necks, the thrombi cannot escape due to the filtering action by the fluid-permeable tube 10 or balloon 30 as described above.

### Further Technical Information

1. Laser light can be administered in the range of 500 to 600 nanometers. If an argon ion laser at 514 nm is used, the light absorption dye can be Erythrocin B which has a peak absorption coefficient to 537 nm. The dose of the dye is 20 mg/kg body weight, if administered systematically; but if flushed through the catheter, the dye load can be reduced. If laser light at 562 nm is used, then the light absorbing dye can be Rose Bengal at the same concentration as the Erythrocin B.

2. It is believed the mechanism of action of the photo thrombosis is that the dye absorbs the light energy and creates the radical singled oxygen which is toxic to the endothelial cells, damages them, and activates the platelets, creating a growing platelet thrombus having a size which depends on the dose of irradiation.

3. Past experience with irradiation in arteries suggests that the input power should be about 200-250 mW, and the normal irradiation time should be about 2-3 minutes.

4. The technical problems with the optical fiber are:
a. The need to disperse the light as it exits from the optical fiber, requiring a convex lens which is not easy to make in a fiber. A diffuser can be used instead.
b. Multimode fibers are very flexible and difficult to push through a microcatheter. The fiber wall needs to be coated with a stiffer material to give it some structural rigidity.

While the invention has been described with respect to several preferred embodiments, it will be appreciated that these are set forth merely for purposes of example, and that many other variations, modifications and applications of the invention may be made.

What is claimed is:

1. A method of reducing or blocking blood flow to a selected blood vessel, or a selected part of a wall thereof, particularly for treating an aneurysm, an arteriovenous malformation, or a dural malformation, or for devascularizing a blood vessel feeding the tumor, such method comprising:
deploying in the selected blood vessel an expandable member having a contracted condition for manipulation within the blood vessel, and expandable to an expanded condition in the blood vessel for reducing or blocking blood flow through the blood vessel or the selected part of the wall thereof, and thereby to promote coagulation of blood within the selected blood vessel or part of the wall thereof;
and applying a local stimulus to the interior of the selected blood vessel or part of the wall thereof effective to initiate or accelerate coagulation of blood therein.

2. The method according to claim 1, wherein said expandable member is a permeable mesh-like tube of biocompatible material dimensioned, when expanded, to straddle the selected part of the blood vessel wall such as to reduce blood flow thereto, and also to prevent thrombus from being swept downstream thereof.

3. The method according to claim 1, wherein said expandable member is an inflatable balloon effective, when inflated, to block blood flow to the selected blood vessel or selected part of the wall thereof, and to prevent thrombus from being swept downstream thereof.

4. The method according to claim 3, wherein said inflatable balloon is effective, when inflated, to straddle the opposite sides of the selected part of the blood vessel or wall thereof.

5. The method according to claim 3, wherein said inflatable balloon is deployed downstream of the selected part of the blood vessel or wall thereof when inflated.

6. The method according to claim 1, wherein said local stimulus is light energy applied by an optical fiber having a tip deployed in the selected part of the blood vessel in which coagulation is to be initiated or accelerated.

7. The method according to claim 6, wherein a light-energy absorption agent is applied to the interior of said selected part of the blood vessel before said light energy is applied thereto.

8. The method according to claim 6, wherein said expandable member is a permeable mesh-like tube of biocompatible material dimensioned, when expanded, to straddle the selected part of the blood vessel wall such as to reduce blood flow thereto; and wherein said optical fiber tip is deployed in the selected part of the blood vessel where coagulation is to be initiated or accelerated, after the partial deployment of the permeable mesh-like tube in said blood vessel.

9. The method according to claim 6, wherein said expandable member is a permeable mesh-like tube of biocompatible material dimensioned, when expanded, to straddle the selected part of the blood vessel wall such as to reduce blood flow thereto; and wherein said optical fiber tip is deployed in the selected part of the blood vessel where coagulation is to be initiated or accelerated, before the full deployment of the permeable mesh-like tube in said blood vessel.

10. The method according to claim 6, wherein said expandable member is a permeable mesh-like tube of biocompatible material dimensioned, when expanded, to straddle the selected part of the blood vessel such as to reduce blood flow thereto; and wherein said optical fiber tip is deployed in the selected part of the blood vessel where coagulation is to be initiated or accelerated, after the full deployment of the permeable mesh-like tube in said blood vessel.

11. The method according to claim 6, wherein said expandable member is a permeable mesh-like tube of biocompatible material dimensioned, when expanded, to straddle the selected part of the blood vessel such as to reduce blood flow thereto; and wherein said permeable mesh-like tube is an expansible tube having an initial contracted state for enabling moving the tube to the site of deployment in the blood vessel, and an expanded state for fixing the tube within the blood vessel.

12. The method according to claim 1, wherein:
said permeable mesh-like tube, while in the contracted state, is moved through the blood vessel to a position wherein its opposite sides straddle the selected part of the blood vessel in which the coagulation is to be initiated or accelerated;
the side of the permeable mesh-like tube facing the downstream direction is expanded;
the optic fiber tip is deployed into the selected part of the blood vessel wall in which coagulation is to be initiated or accelerated;
and then light energy is applied to the optical fiber to cause its tip to initiate or accelerate coagulation of blood therein while the permeable mesh-like tube prevents emboli resulting from said coagulation from moving through the blood vessel in the downstream direction.

13. The method according to claim 12, wherein the side of the permeable mesh-like tube facing the upstream direction is expanded after the deployment of the optical fiber tip in said selected part of the blood vessel.

14. The method according to claim 1, wherein:
said permeable mesh-like tube, while in the contracted state, is moved through the blood vessel to a position wherein its opposite sides straddle the opposite sides of the selected part of the blood vessel in which coagulation is to be initiated or accelerated;
said optic fiber tip is deployed into the selected part of the blood vessel where coagulation is to be initiated or accelerated, by moving the optical fiber between the outer surface of the permeable mesh-like tube and the inner surface of the blood vessel;
said permeable mesh-like tube is then expanded to fix it within the blood vessel straddling the selected part of the blood vessel;
and light energy is then applied to the optical fiber to initiate or accelerate coagulation of the blood within the selected part of the blood vessel wall, while the permeable mesh-like tube prevents emboli resulting from said coagulation from moving through the tube into the blood vessel.

15. The method according to claim 11, wherein
said permeable mesh-like tube, while in the contracted state, is moved through the blood vessel to a position wherein its opposite sides straddle the selected part of the blood vessel in which the coagulation is to be initiated or accelerated;
the permeable mesh-like tube is expanded to fix it within the blood vessel and;
said optical fiber tip is then deployed by moving the optical fiber through the interior of the expanded permeable mesh-like tube with its tip passing through the permeable mesh-like tube into said selected part of the blood vessel where coagulation is to be initiated or accelerated.

16. The method according to claim 6, wherein said optical fiber tip is deployed into said selected part of the blood vessel via a microcatheter.

17. The method according to claim 16, wherein said microcatheter for deploying said optical fiber tip into said selected part of the blood vessel is also used for applying a light-energy absorption agent into said selected part of the blood vessel before applying said light energy thereto.

18. The method according to claim 11, wherein said permeable mesh-like tube includes an outer sheath normally constraining the permeable mesh-like tube to its contracted state, which sheath is removable to permit the tube to expand to its expanded state.

19. The method according to claim 10, wherein said optical fiber tip includes a surface on its end and lateral sides for diffusing light energy around said tip.

20. The method according to claim 19, wherein said light energy is laser energy.

21. The method according to claim 1, wherein said local stimulus is a pharmacological agent which induces thrombosis.

22. A kit for use in reducing or blocking blood flow to a selected blood vessel, or a selected part or a wall thereof, particularly for treating an aneurysm, an arteriovenous malformation, or a dural malformation, or for devascularizing a blood vessel feeding a tumor, said kit comprising:
an expandable member having a contracted condition for manipulation within the blood vessel and expandable to an expanded condition within the blood vessel for reducing or blocking blood flow to the selected part of the blood vessel or wall thereof, and thereby to promote coagulation of blood therein;
and a local stimulus applicator for applying a local stimulus to the interior of the selected part of the blood vessel, such as to initiate or accelerate coagulation of blood therein.

23. The kit according to claim 22, wherein said expandable member is a permeable mesh-like tube of biocompatible material dimensioned, when expanded, to straddle the selected part of the blood vessel or wall thereof, such as to reduce blood flow thereto.

24. The method according to claim 1, wherein said expandable member is an inflatable balloon effective, when inflated, to block blood flow to the selected blood vessel or selected part of the wall thereof.

25. The kit according to claim 22, wherein said local stimulus applicator is an optical fiber having a tip to be located within said selected part of the blood vessel in which coagulation is to be initiated or accelerated.

26. The kit according to claim 25, further including a microcatheter for deploying said optical fiber tip.

27. The kit according to claim 26, wherein said microcatheter comprises:
an optical fiber having a tip including diffusive surfaces on its end and lateral sides for emitting light energy around the tip;
a catheter tube enclosing said optical fiber for deploying said optical fiber tip into the selected part of the blood vessel in which coagulation is to be initiated or accelerated;
and an applicator for delivering to the interior of said selected part of the blood vessel, before said light energy is applied thereto, a light-energy absorption agent via space between said optic fiber and said catheter tube.

28. The microcatheter according to claim 27, wherein said optical fiber includes a convex diffusive cap at said tip.

29. A microcatheter particularly useful in the method of claim 1, said microcatheter comprising:
an optical fiber having a tip including diffusive surfaces on its end and lateral sides for emitting light energy around the tip;
a catheter tube enclosing said optical fiber for deploying said optical fiber tip into the selected part of the blood vessel in which coagulation is to be initiated or accelerated;
and an applicator for delivering to the interior of said selected part of the blood vessel, before said light energy is applied thereto, a light-energy absorption agent via space between said optic fiber and said catheter tube.

30. The microcatheter according to claim 28, wherein said optical fiber includes a convex diffusive cap at said tip.
